(19) United States
(12) Patent Application Publication
(10) Pub. No.: US 2005/0084473 A1
(43) Pub. Date: Apr. 21, 2005

(54) Treatment of acne by topical herbal extract solution

(76) Inventor: William Lee, Corona Del Mar, CA (US)

Correspondence Address: WILLIAM LEE, 341 EVENING CANYIN, CORONA DEL MAR, CA, 92625, US

(21) Appl. No.: 10/688,227

(22) Filed: Oct. 20, 2003

(51) Int. Cl. A61K 7/06; A61K 35/78

(52) U.S. Cl. 424/74; 424/725; 424/769

(57) ABSTRACT

A topical solution (may be a cream or gel or liquid) is disclosed for the treatment of acne lesion. The topical solution contains herbal extracts Dandelion (Taraxaci Mongolici Cum Radice Herba) in powder form approx. 0.5-5%, Senecio (Senecionis Scandens Herba) in powder form approx. 0.1-6%, Elephantopi (Elephantopus Scaber Herba) in powder form approx. 0.1-5%, Lonicera (Lonicerae Japanicae Flos) in powder form approx. 0.1-4%, Angelica (Fragrant Angelica) in powder form approx. 0.1-5%, Scute (Scutellariae Baicalensis Radix) in powder form approx 0.1-5%, Witch Hazel (Hamamelis Virginiana L.) in liquid form approx. 0.1-6% provides the action of killing bacteria, removal of inflammation and loosening of the blackheads of comedones in the pilosebaceous unit. The solution also contains transdermal agents includes Benzyl Alcohol approx. 0.1-5%, Propylene Glycol approx. 0.1-10%, Ethyl Alcohol 70% approx. 1-20% to facilitate penetration. The invention relates to herbal extract solution with transdermal agents for the use in the topical treatment of acne and other related inflammatory ailments. The invention further relates to methods of making and using the composition for the treatment of acne lesion.

This invention relates to the development, formulation and application of a topical herbal extract solution for the treatment of acne.

### FIELD OF THE INVENTION

The present invention relates to a topical herbal extract solution for the treatment of acne (comedones, pimples) and cystic acne. The invention also relates to the field of composition, method of making and using the composition for the treatment of acne and related ailments.

### BACKGROUND OF THE INVENTION

Acne is an inflammatory follicular, papular, and pustular eruption involving the pilosebaceous apparatus. Androgen stimulates sebaceous glands to produce large amounts of sebum, bacteria contain lipases that convert lipids to fatty acids. Both sebum and fatty acids cause a sterile inflammatory response in the pilosebaceous unit, which results in hyperkeratinization of the lining of the follicle with resultant plugging. The enlarged follicular lumen contains this inspissated keratin and lipid debris (white heads). When the follicle has a portal of entry at the skin forming a plug, it is termed a black head. The distended follicular wall may break and exude its contents (sebum, lipids, fatty acids, keratin etc). Bacteria may enter the dermis, provoking a foreign body response (papule, pustule, nodule). Currently, oral antibiotics and vitamin A related compounds are the most common medications prescribed by physicians to treat acne. Unfortunately, they are not very effective and also cause systemic side effects. Topical antibiotics are also available by prescription but the success of the treatment is low. I see a need for a topical herbal extract, by using natural herbal extracts as active ingredients in combination with pharmaceutical transdermal agents that can be used topically. The overall outcome is to provide successful treatment with none or very minimal side effects.

### SUMMARY OF THE INVENTION

The present invention relates to an herbal extract solution with three transdermal pharmaceutical agents for the treatment of acne. The present invention contains seven herbal extracts:

1. Dandelion (Aramaic Mongolic Cum Radices Herbal) in powder form approx. 0.5-5%

2. Seneca (Selections Scandens Herba) in powder form approx. 0.1-6%

3. Elephantopi (Elephantopus Scaber Herba) in powder form approx. 0.1-5%

4. Lonicera (Lonicerae Japanicae Flos) in powder form approx. 0.1-4%

5. Angelica (Fragrant Angelica) in powder form approx. 0.1-5%

6. Scute (Scutellariae Baicalensis Radix) in powder form approx 0.1-5%

7. Witch Hazel (Hamamelis Virginiana L.) in liquid form approx. 0.1-6%

The present invention contains seven herbal extracts each of which exists in powder or liquid form. The present invention relates to an herbal extract solution, which also contains three transdermal agents:

1. Benzyl Alcohol approx. 0.1-5%

2. Propylene Glycol approx. 0.1-10%

3. Ethyl Alcohol 70% approx. 1-20%

The seven herbal extracts are either in liquid or powder form first weighed and dissolved in distilled water at 60 degrees Celsius thoroughly mixed and allowed to cool to 40 degrees Celsius. The transdermal agents are added to the herbal extracts with distilled water. The contents are mixed for 30 minutes and left for 12 hours at room temperature. The resultant clear solution will be obtained by filtration. The invention is applied using a cotton swab to the acne area. This invention can cure acne by destroying the bacteria in the hair follicle and sebaceous glands and it can also remove the inflammatory elements from the pilosebaceous unit, within 3 days to 5 days, there is a unique action with this invention that can loosen the black head of acne and allow the transdermal agents to carry the herbal extract in vapor flux form down to the hair follicle and sebaceous gland to kill the bacteria and remove the inflammatory elements.

The invention is related to the field of composition and method for the treatment of acne. This invention further relates to the methods of making and using this composition for the treatment of acne lesion includes all the herbal extracts and the transdermal agents as well as distilled water mentioned in the Summary of the Invention.

It is the objective of this invention to provide a novel herbal extract solution for the topical treatment of acne lesions, by providing a formulation of herbal extracts, transdermal agents, and water, the formulation creates a vapor flux penetration of the herbal extracts, which loosen black heads of acne and treat the acne by killing the bacteria and removing the inflammatory elements in the pilosebaceous unit.

Although the invention has been described herein by reference to specific embodiments and example, it is to be understood that this disclosure, as read by one skilled in the art, is much broader than these specific embodiments, and should not be limited in any way beyond the scope of the following claims.

### DETAILED DESCRIPTION

We disclose herein:

1. Herbal anti-inflammatory/anti-infectious agents

2. Methods of preparing the final herbal extract solution

3. Preparing and using herbal anti-inflammatory/anti-infectious agents with transdermal agents for topical preparation.

The topical solution has anti-inflammatory/anti-infectious properties for the treatment of acne lesions as disclosed herein is prepared according to the following procedure:

1. Seven different extracts either in powder or liquid form or natural form are dissolved in distilled water at 60 degrees Celsius.

2. After mixed thoroughly the herbal extract and water will cool down to 40 degrees Celsius

3. At 40 degrees the three transdermal agents are added to the herbal extract solution and water mixed thoroughly for 30 minutes.

The final solution will contain herbal extract, water and transdermal agents will be left alone in a container for 12 hours for further self extraction by the transdermal agent, then followed by filtering, the final solution will be clear and ready for application. Xanthan gum 0.1%-5% may add to thicken the solution. One may also try to increase the anti-inflammatory action of the above invention by adding Salicylic acid 0.1%-5%.

1. A topical solution for the treatment of acne lesion comprising: Dandelion (Taraxaci Mongolici Cum Radice Herba) in powder form approx. 0.5-5%, Senecio (Senecionis Scandens Herba) in powder form approx. 0.1-6%, Elephantopi (Elephantopus Scaber Herba) in powder form approx. 0.1-5%, Lonicera (Lonicerae Japanicae Flos) in powder form approx. 0.1-4%, Angelica (Fragrant Angelica) in powder form approx. 0.1-5%, Scute (Scutellariae Baicalensis Radix) in powder form approx 0.1-5%, Witch Hazel (Hamamelis Virginiana L.) in liquid form approx. 0.1-6%, for treating infection by killing bacteria in the hair follicle and sebaceous gland, removing the inflammation in the hair follicle and sebaceous gland, and loosening the black head of the comedones. Fast penetration of the herbal extract in vapor flux form is provided by the transdermal system. The transdermal system contains three agents to facilitate dermal penetration includes Benzyl Alcohol approx. 0.1-5%, Propylene Glycol approx. 0.1-10%, Ethyl Alcohol 70% approx. 1-20%.

2. The topical solution of claim 1. Comprised of an herbal extract containing ethyl alcohol, benzyl alcohol, propylene glycol, and water.

3. A method for treating a patient having acne lesions comprises the steps of: providing a topical solution that is defined in claim 1 and applying the solution to the area with the acne lesion.

4. A method for making the topical solution is outlines above in the Detailed Description.
